(19) United States
(12) Patent Application Publication
(10) Pub. No.: US 2024/0206825 A1
(43) Pub. Date: Jun. 27, 2024

(54) ESTIMATION DEVICE, ESTIMATION SYSTEM, ESTIMATION METHOD, AND STORAGE MEDIUM

(71) Applicant: NEC Corporation, Minato-ku, Tokyo (JP)

(72) Inventor: Takeo NOZAKI, Tokyo (JP)

(73) Assignee: NEC Corporation, Minato-ku, Tokyo (JP)

(21) Appl. No.: 18/288,090

(22) PCT Filed: May 7, 2021
(86) PCT No.: PCT/JP2021/017463

§ 371 Date: Oct. 24, 2023

(51) Int. Cl. A61B 5/00; A61B 5/024; A63B 24/00; G16H 20/30

(52) U.S. Cl. CPC A61B 5/7278; A61B 5/02405; A63B 24/0062; G16H 20/30; A63B 2230/065

(57) ABSTRACT

An estimation device according to an aspect of the present disclosure includes: at least one memory storing a set of instructions; and at least one processor configured to execute the set of instructions to: receive a transition of a heart rate of a target person in a state including a resting state and an active state, the transition of the heart rate being measured by a heart rate measurement device; estimate a fatigue level of the target person based on an estimation model and the transition of the heart rate, the estimation model estimating the fatigue level based on the heart rate; and output the fatigue level.

### TECHNICAL FIELD

The present disclosure relates to a technique for estimating a heart rate.

### BACKGROUND ART

A technique for estimating a fatigue level representing a degree of fatigue of a person is disclosed in, for example, the following document.

PTL 1 describes a method for calculating a fatigue level based on accumulation of an exercise load. In the fatigue level described in PTL 1, the fatigue level on a past day attenuates at an attenuation rate based on the number of elapsed days from the relevant day and the number of rest days between the relevant days.

PTL 2 discloses a method for calculating the fatigue level from a brain fatigue level based on fluctuations in heartbeat intervals and a physical fatigue level based on a difference between a lying heart rate measured in a state of facing upward and a standing heart rate measured in a state of standing.

### CITATION LIST

### Patent Literature

PTL 1: JP 2018-033565 A
PTL 2: JP 2017-063963 A

### SUMMARY OF INVENTION

### Technical Problem

In the method of Patent Document 1, an exercise load needs to be obtained in order to calculate the fatigue level. Furthermore, in the method of Cited Document 1, the fatigue level cannot be calculated at short time intervals. In the method of Patent Document 2, the lying heart rate and the standing heart rate need to be measured in order to calculate the fatigue level. In other words, the target person to calculate the fatigue level needs to perform a specific operation (i.e., in this example, the operation of lying down and the operation of standing up) in order to calculate the fatigue level. Therefore, in order to calculate the fatigue level, a time to measure the heart rate in the lying position and to further measure the heart rate in the standing position is at least required.

One object of the present disclosure is to provide an estimation device and the like capable of improving the real-time property of the estimation of the fatigue level.

### Solution to Problem

An estimation device according to one aspect of the present disclosure includes: reception means for receiving a transition of a heart rate of a target person in a state including a resting state and an active state, the transition of the heart rate being measured by a heart rate measurement device; fatigue level estimation means for estimating the fatigue level of the target person based on an estimation model and the transition of the heart rate, the estimation model estimating the fatigue level based on the heart rate; and output means for outputting the fatigue level.

An estimation method according to one aspect of the present disclosure includes: receiving a transition of a heart rate of a target person in a state including a resting state and an active state, the transition of the heart rate being measured by a heart rate measurement device; estimating a fatigue level of the target person based on an estimation model and the transition of the heart rate, the estimation model estimating a fatigue level based on a heart rate; and outputting the fatigue level.

A storage medium according to one aspect of the present disclosure stores a program that causes a computer to execute: reception processing of receiving a transition of a heart rate of a target person in a state including a resting state and an active state, the transition of the heart rate being measured by a heart rate measurement device; fatigue level estimation processing of estimating a fatigue level of the target person based on an estimation model and the transition of the heart rate, the estimation model estimating a fatigue level based on a heart rate; and outputting the fatigue level. The present disclosure is also achieved by the above-described program according to an aspect of the present disclosure.

### Advantageous Effects of Invention

The present disclosure has an effect of improving the real-time property of the estimation of the fatigue level.

### BRIEF DESCRIPTION OF DRAWINGS

FIG. 1 is a block diagram illustrating an example of a configuration of an estimation device 10 according to a first example embodiment of the present disclosure.

FIG. 2 is a flowchart illustrating an example of the operation of the estimation device 10 according to the first example embodiment of the present disclosure.

FIG. 3 is a block diagram illustrating an example of a configuration of an estimation system 1 according to a second example embodiment of the present disclosure.

FIG. 4 is a block diagram schematically illustrating an example of implementation of an estimation system 1 according to a second example embodiment of the present disclosure.

FIG. 5 is a diagram schematically illustrating an example of transition of the heart rate received by a reception unit 110 according to the second example embodiment of the present disclosure.

FIG. 6 is a flowchart illustrating an example of the operation of an estimation device 100 according to the second example embodiment of the present disclosure.

FIG. 7 is a flowchart illustrating an example of the operation of an estimation device 100 according to a modified example of the second example embodiment of the present disclosure.

FIG. 8 is a flowchart illustrating an operation of the receiving process of the estimation device 100 according to the second example embodiment of the present disclosure.

FIG. 9 is a diagram illustrating an example of Borg index with respect to the estimated heart rate.

FIG. 10 is a diagram illustrating an example of a relationship between the measured maximum heart rate and the heart-lung capacity achievement ratio.

FIG. 11 is a diagram illustrating another example of the relationship between the measured maximum heart rate and the heart-lung capacity achievement ratio.

FIG. 12 is a diagram illustrating transition of heart rate and transition of exercise intensity.

FIG. 13 is a diagram illustrating transition of heart rate and transition of exercise intensity.

FIG. 14 is a diagram illustrating an example of a transition of the heart rate and an example of a straight line of the calculated slope.

FIG. 15 is a diagram illustrating an example of a hardware configuration of a computer 1000 that can realize each of the estimation devices according to the example embodiments of the present disclosure.

### EXAMPLE EMBODIMENT

Hereinafter, example embodiments of the present disclosure will be described in detail using the drawings.

### First Example Embodiment

First, a first example embodiment of the present disclosure will be described.

### <Configuration>

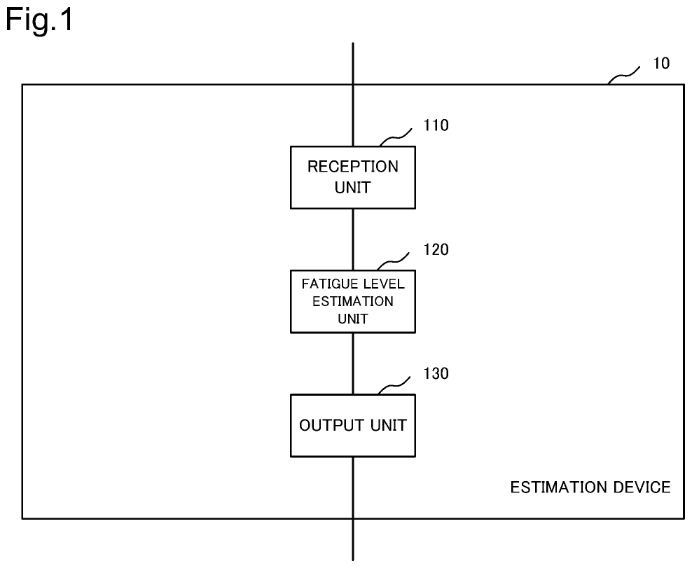

FIG. 1 is a block diagram illustrating an example of a configuration of an estimation device 10 according to a first example embodiment of the present disclosure. In the example illustrated in FIG. 1, the estimation device 10 includes a reception unit 110, a fatigue level estimation unit 120, and an output unit 130. The reception unit 110 receives the transition of the heart rate of a target person in the state including a resting state and an active state measured by a heart rate measurement device. The fatigue level estimation unit 120 estimates the fatigue level of the target person based on an estimation model that estimates the fatigue level based on the heart rate and a transition of the heart rate. The output unit 130 outputs the fatigue level.

The heart rate measurement device is, for example, a device that measures a heart rate included in a wearable terminal. The transition of the heart rate is, for example, time-series data of the heart rate per unit time calculated every predetermined time. The transition of the heart rate is time series data of the heart rate measured in at least one period. Each heart rate (specifically, the value of the heart rate) included in the transition of the heart rate may be associated with a value representing the time when the heart rate is measured. If the transition of heart rate is time series data of the heart rate measured in two or more periods, at least one period may include a plurality of measurement time points and the other period may include only one measurement time point. For example, the fatigue level estimation unit 120 estimates the fatigue level at the time point when the heart rate included in the transition of the heart rate is measured. A method of estimating the fatigue level by the estimation model and the transition of the heart rate will be described in detail later.

### <Operation>

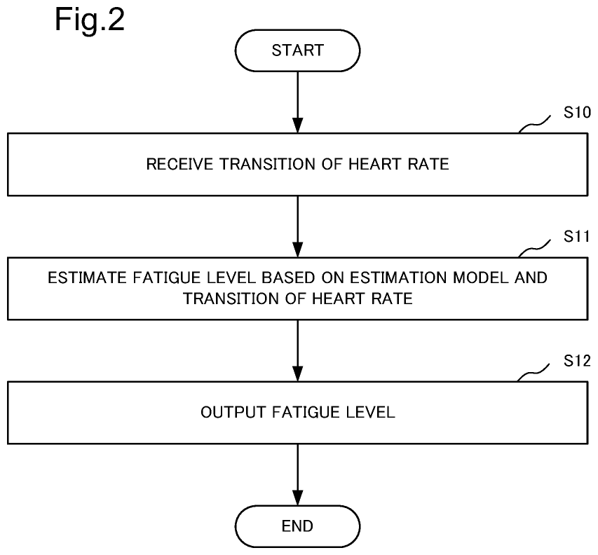

FIG. 2 is a flowchart illustrating an example of the operation of the estimation device 10 according to the first example embodiment of the present disclosure. In the example illustrated in FIG. 2, the reception unit 110 receives the transition of the heart rate (step S10). In other words, the reception unit 110 receives information representing the transition of the heart rate. Next, the fatigue level estimation unit 120 estimates the fatigue level based on the estimation model and the transition of the heart rate (step S11). Then, the output unit 130 outputs the estimated fatigue level (step S12).

### <Effects>

The present example embodiment has an effect of improving the real-time property of the estimation of the fatigue level. This is because the fatigue level estimation unit 120 estimates the fatigue level based on the estimation model and the transition of the heart rate of the target person in the state including the resting state and the active state. If the transition of the heart rate of the target person in the state including the resting state and the active state is obtained, a new measurement of the heart rate is not necessary to estimate the fatigue level. Therefore, the estimation device 10 of the present example embodiment can improve the real-time property of the estimation of the fatigue level.

### Second Example Embodiment

Next, a second example embodiment of the present disclosure will be described in detail with reference to the drawings.

### <Configuration>

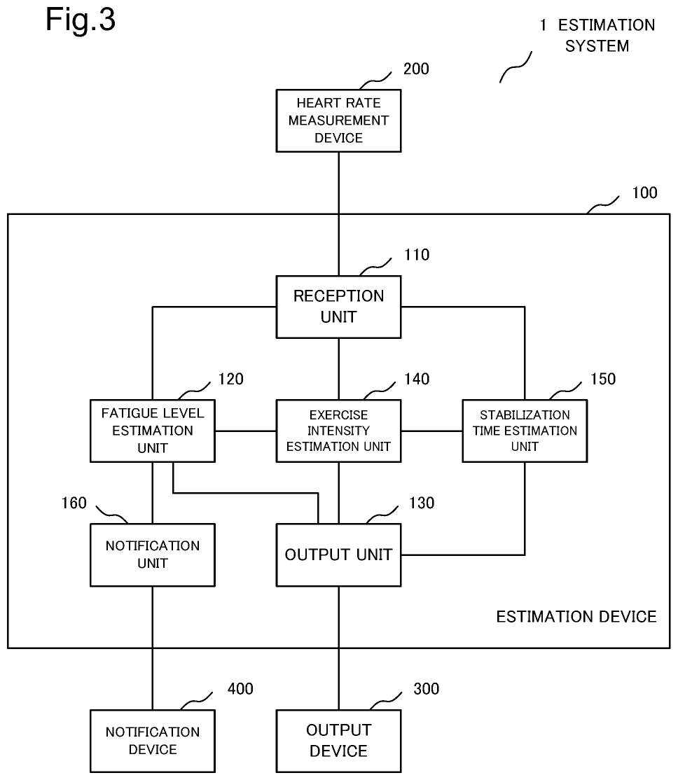

FIG. 3 is a block diagram illustrating an example of a configuration of an estimation system 1 according to a second example embodiment of the present disclosure. In the example illustrated in FIG. 3, the estimation system 1 includes an estimation device 100, a heart rate measurement device 200, an output device 300, and a notification device 400. The estimation device 100 is communicably connected to the heart rate measurement device 200, the output device 300, and the notification device 400.

### <Heart Rate Measurement Device 200>

The heart rate measurement device 200 is, for example, a heart rate meter that measures a heart rate per unit time and outputs the measured heart rate per unit time, for example, every predetermined time. The heart rate measurement device 200 may transmit the measured heart rate to the estimation device 100 each time the heart rate is measured. The heart rate measurement device 200 may transmit the heart rate obtained by two or more measurements to the estimation device 100 at one time. In the following description, the heart rate measurement device 200 transmits the measured heart rate to the estimation device 100 each time the heart rate is measured. In addition, a time series of a plurality of heart rates measured from the start to the interruption of the measurement of the heart rate by the heart rate measurement device 200 is indicated as a transition of the heart rate. As described above, the number of heart rates included in the transition of the heart rate may be one or two or more.

### <Output Device 300>

The output device 300 is, for example, a display. The output device 300 may be an information processing device such as a computer or a server different from the estimation device 100. The output device 300 may be a terminal device that allows an administrator who manages the target person to view the screen.

### <Notification Device 400>

The notification device 400 is a device that notifies the target person whose heart rate is measured by the heart rate measurement device 200. The notification device 400 includes a display, a speaker, a vibrator, a light emitting element, and the like. The notification is performed by at least any one of sound, light, vibration, text, image, or the like using a display, a speaker, a vibrator, a light emitting element, or the like.

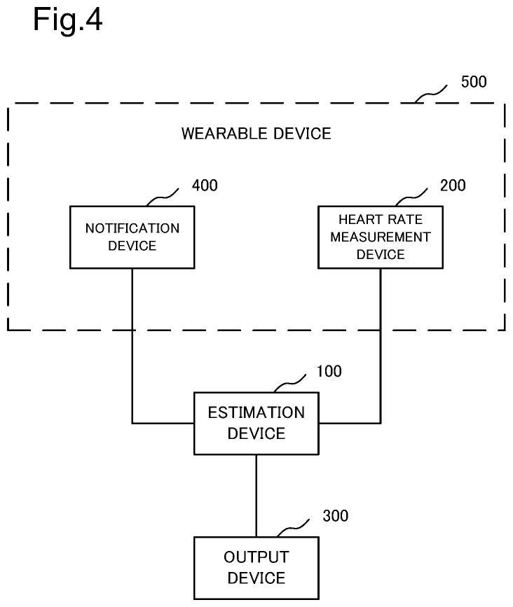

FIG. 4 is a block diagram schematically illustrating an example of implementation of the estimation system 1 according to the second example embodiment of the present disclosure. In the example illustrated in FIG. 4, the heart rate measurement device 200 and the notification device 400 described above are included in a wearable device 500. The heart rate measurement device 200 measures the heart rate of the target person wearing the wearable device 500. The notification device 400 notifies the target person to whom the wearable device 500 is to be worn. The wearable device 500 may be a walking assist robot. In the example illustrated in FIG. 4, the output device 300 is not included in the wearable device 500, but the output device 300 may also be included in the wearable device 500. Then, the target person may be able to confirm the output by the output device 300. In this case, the output device 300 may operate as a notification device.

The notification device 400 may notify an administrator who manages the target person instead of the target person. In this case, the notification device 400 may be, for example, a terminal device held by an administrator. The notification device 400 may be, for example, a terminal device that allows an administrator to view a screen.

### <Estimation Device 100>

In the example illustrated in FIG. 3, the estimation device 100 includes a reception unit 110, a fatigue level estimation unit 120, an output unit 130, an exercise intensity estimation unit 140, a stabilization time estimation unit 150, and a notification unit 160.

### <Reception Unit 110>

The reception unit 110 receives from the heart rate measurement device 200 the transition of the heart rate of a target person in the state including a resting state and an active state measured by a heart rate measurement device 200. As described above, the transition of the heart rate received by the reception unit 110 is the transition of the heart rate of the target person in a state in which the measured resting state and active state are included in at least one period. In a case where the reception unit 110 receives the transition of the heart rate in a plurality of periods, the transition of the heart rate in one or more periods may not include the transition of the heart rate measured in a state including the resting state and the active state. The transition of the heart rate not including the transition of the heart rate measured in the state including the resting state and the active state may include only the heart rate measured at one time point.

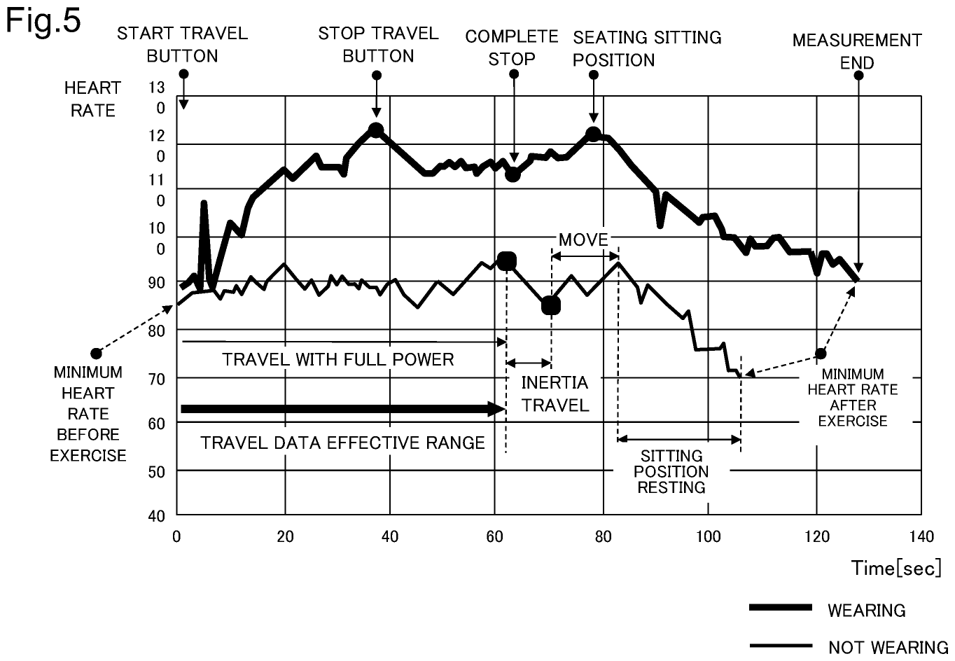

FIG. 5 is a diagram schematically illustrating an example of transition of the heart rate received by the reception unit 110 according to the second example embodiment of the present disclosure. FIG. 5 illustrates a transition of the heart rate measured in a state where the target person wears the robot suit, and a transition of the heart rate measured in a state where the target person does not wear the robot suit but wears the heart rate measurement device 200.

The reception unit 110 sends the transition of the received heart rate (specifically, information indicating the transition of the heart rate) to the fatigue level estimation unit 120.

### <Fatigue Level Estimation Unit 120>

The fatigue level estimation unit 120 receives the transition of the heart rate (specifically, information indicating the transition of the heart rate) from the reception unit 110. The fatigue level estimation unit 120 estimates the fatigue level of the target person based on an estimation model that estimates the fatigue level based on the heart rate and a transition of the heart rate. Specifically, the fatigue level estimation unit 120 detects the measured maximum heart rate, which is the maximum heart rate in the transition of the heart rate, and the resting heart rate, which is the heart rate in the resting state, from the transition of the received heart rate. The resting heart rate is, for example, the minimum heart rate in the transition of the heart rate. The fatigue level estimation unit 120 estimates the fatigue level at the fatigue level estimation target time point based on the measured maximum heart rate, the resting heart rate, the heart rate at the fatigue level estimation target time point, and the estimation model. A fatigue level and a specific method of calculating the fatigue level will be described in detail later.

The fatigue level estimation target time point may be a time point when the latest heart rate is measured in the transition of the heart rate measured by the heart rate measurement device 200.

The fatigue level estimation unit 120 sends the estimated fatigue level to the output unit 130. The fatigue level estimation unit 120 may associate the time of the fatigue level estimation target time point with the estimated fatigue level, and send the fatigue level associated with the time of the fatigue level estimation target time point to the output unit 130.

The fatigue level estimation unit 120 sends the estimated fatigue level to the notification unit 160. The fatigue level estimation unit 120 may associate the time of the fatigue level estimation target time point with the estimated fatigue level, and send the fatigue level associated with the time of the fatigue level estimation target time point to the notification unit 160.

The fatigue level estimation unit 120 sends the transition of the heart rate and the information indicating the fatigue level estimation target time point to the exercise intensity estimation unit 140. Furthermore, the fatigue level estimation unit 120 sends information indicating the measured maximum heart rate and information indicating the resting heart rate to the exercise intensity estimation unit 140. The information indicating the measured maximum heart rate is, for example, a value of the measured maximum heart rate and information specifying the time when the measured maximum heart rate is measured. The information indicating the resting heart rate is, for example, a value of the resting heart rate and information specifying a time when the resting heart rate is measured. The fatigue level estimation unit 120 may send information indicating the heart rate at the fatigue level estimation target time point to the exercise intensity estimation unit 140 instead of the transition of the heart rate and the information indicating the fatigue level estimation target time point. The information indicating the heart rate at the fatigue level estimation target time point may be the transition of the heart rate and information indicating the fatigue level estimation target time point.

### <Exercise Intensity Estimation Unit 140>

The exercise intensity estimation unit 140 receives, from the fatigue level estimation unit 120, the transition of the heart rate, the information indicating the fatigue level estimation target time point, the information indicating the measured maximum heart rate, and the information indicating the resting heart rate. The exercise intensity estimation unit 140 may receive, from the fatigue level estimation unit 120, information indicating the heart rate at the fatigue level estimation target time point instead of the transition of the heart rate and the information indicating the fatigue level estimation target time point.

The exercise intensity estimation unit 140 estimates the exercise intensity at an intensity estimation target time point based on the transition of the heart rate. Specifically, the exercise intensity estimation unit 140 estimates the exercise intensity at the intensity estimation target time point based on the measured maximum heart rate, the resting heart rate, and the heart rate at the intensity estimation target time point. An exercise intensity and a method of estimating the exercise intensity will be described in detail later.

The strength estimation target time point may be appropriately designated. The intensity estimation target time point may be the same as the fatigue level estimation target time point. In the following description, the intensity estimation target time point is the same as the fatigue level estimation target time point.

The exercise intensity estimation unit 140 sends the estimated exercise intensity to the output unit 130.

The exercise intensity estimation unit 140 may send the transition of the heart rate, the information indicating the fatigue level estimation target time point, the information indicating the measured maximum heart rate, and the information indicating the resting heart rate to the stabilization time estimation unit 150.

### <Stabilization Time Estimation Unit 150>

The stabilization time estimation unit 150 receives, from the exercise intensity estimation unit 140, the transition of the heart rate, the information indicating the fatigue level estimation target time point, the information indicating the measured maximum heart rate, and the information indicating the resting heart rate.

The stabilization time estimation unit 150 estimates the stabilization time based on the transition of the heart rate. The stabilization time is, for example, a time from the stabilization time estimation target time point until the heart rate of the target person becomes the sitting position stable state when the state of the target person transitions to the sitting position stable state at the stabilization time estimation target time point. The stabilization time estimation unit 150 may set the sitting position stable state to a state in which the heart rate of the target person becomes the resting heart rate. The stabilization time estimation unit 150 may set the fatigue level estimation target time point as the stabilization time estimation target time point. A method of estimating the stabilization time will be described in detail later.

The stabilization time estimation unit 150 sends the estimated stabilization time to the output unit 130.

### <Output Unit 130>

The output unit 130 receives the fatigue level from the fatigue level estimation unit 120. The output unit 130 receives the exercise intensity from the exercise intensity estimation unit 140. The output unit 130 receives the stabilization time from the stabilization time estimation unit 150.

The output unit 130 outputs the received fatigue level, exercise intensity, and stabilization time. For example, in a case where the fatigue level estimation target time point is a time when the latest heart rate is measured in the transition of the received heart rate, and the intensity estimation target time point is the same as the fatigue level estimation target time point, the output unit 130 outputs the latest fatigue level and the exercise intensity. In this case, the heart rate measurement device 200 transmits the continuously measured heart rate to the estimation device 100, and the output unit 130 continuously outputs the fatigue level and the exercise intensity, so that the fatigue level and the exercise intensity can be known in real time. In addition, as described above, the stabilization time estimation target time point may be the same as the fatigue level estimation target time point. Then, the output unit 130 may continuously output the stabilization time in addition to the fatigue level and the exercise intensity.

### <Notification Unit 160>

The notification unit 160 receives the fatigue level from the fatigue level estimation unit 120. In a case where the received fatigue level indicates that the fatigue is greater than the predetermined level, the notification unit 160 notifies the target person. Specifically, the notification unit 160 compares the received value of the fatigue level with a threshold value representing a predetermined level to determine whether the received fatigue level indicates that the fatigue is greater than the predetermined level. In a case where the received fatigue level indicates that the fatigue is greater than the predetermined level, the notification unit 160 controls the notification device 400 so that the notification device 400 performs the notification. More specifically, for example, the notification unit 160 transmits an instruction to perform notification to the notification device 400. The notification device 400 that has received the instruction to perform notification notifies the target person.

### <Operation>

Next, an operation of the estimation device 100 according to the second example embodiment of the present disclosure will be described in detail with reference to the drawings.

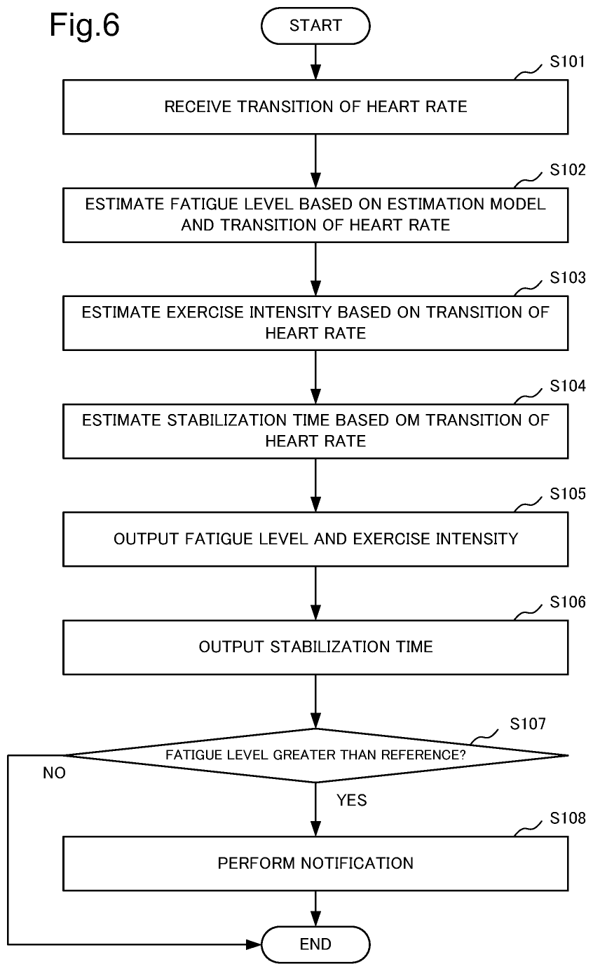

FIG. 6 is a flowchart illustrating an example of the operation of the estimation device 100 according to the second example embodiment of the present disclosure.

In the example illustrated in FIG. 6, the reception unit 110 receives the transition of the heart rate from the heart rate measurement device 200 (step S101). Next, the fatigue level estimation unit 120 estimates the fatigue level based on the estimation model and the transition of the heart rate (step S102). The exercise intensity estimation unit 140 estimates the exercise intensity based on the transition of the heart rate S103). The stabilization time estimation unit 150 estimates the stabilization time based on the transition of the heart rate (S104). Then, the output unit 130 outputs the fatigue level and the exercise intensity (step S105). In addition, the output unit 130 outputs the stabilization time (step S106). The output unit 130 may collectively perform the operation of step S105 and the operation of step S106. When the fatigue level is not greater than the reference (NO in step S107), that is, when the fatigue level does not indicate that the fatigue is greater than the predetermined level, the estimation device 100 ends the operation illustrated in FIG. 6.

When the fatigue level is greater than the reference (YES in step S107), that is, when the fatigue level does not indicate that the fatigue is greater than the predetermined level, the notification unit 160 performs a notification (step S108). Then, the estimation device 100 ends the operation illustrated in FIG. 6.

### <Effects>

The present example embodiment described as abnormal has the same effect as the effect of the first example embodiment. The reason is the same as the reason why the effect of the first example embodiment occurs.

### Modification of Second Example Embodiment

The reception unit 110 may send the transition of the received heart rate (specifically, information indicating the transition of the heart rate) to the exercise intensity estimation unit 140. In this case, the fatigue level estimation unit 120 may not send the transition of the heart rate to the exercise intensity estimation unit 140. The fatigue level estimation unit 120 may not send the measured maximum heart rate and the resting heart rate to the exercise intensity estimation unit 140. In this case, the exercise intensity estimation unit 140 detects the measured maximum heart rate and the resting heart rate in the transition of the received heart rate.

The reception unit 110 may send the transition of the received heart rate (specifically, information indicating the transition of the heart rate) to the stabilization time estimation unit 150. In this case, the exercise intensity estimation unit 140 may not send the transition of the heart rate to the stabilization time estimation unit 150. In addition, the exercise intensity estimation unit 140 may not send the measured maximum heart rate to the stabilization time estimation unit 150. In this case, the stabilization time estimation unit 150 detects the measured maximum heart rate in the transition of the received heart rate.

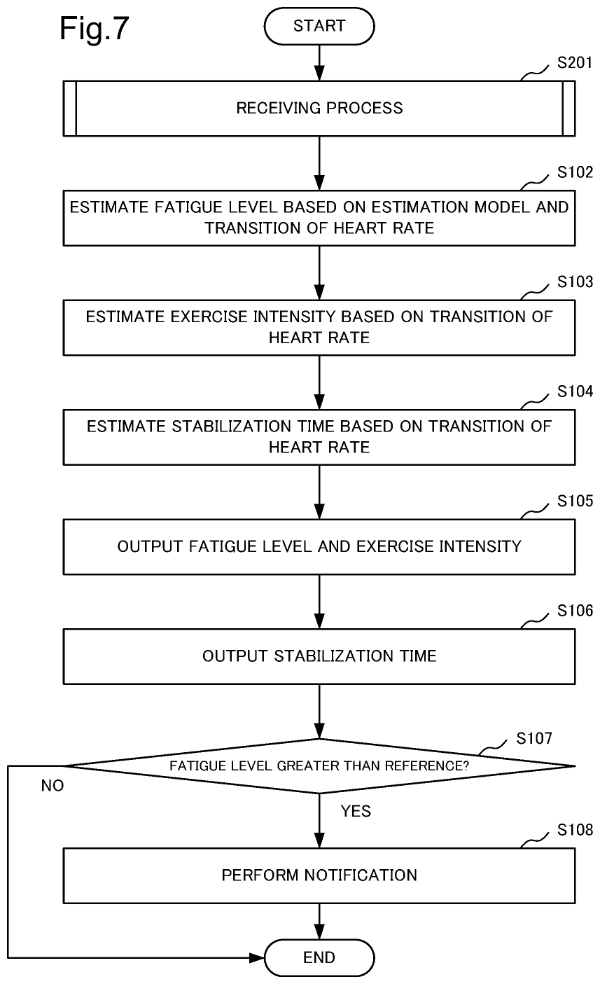

Furthermore, in the operation illustrated in FIG. 6, the reception unit 110 receives the transition of the heart rate in step S101, but for example, the reception unit 110 may continuously receive the heart rate. Then, for example, the fatigue level estimation unit 120 may store the received heart rate. In other words, the fatigue level estimation unit 120 may generate data indicating the transition of the heart rate from the plurality of received heart rates. Then, the fatigue level estimation unit 120 may hold data indicating the transition of the heart rate. When receiving the heart rate, the fatigue level estimation unit 120 may update the transition of the heart rate by adding the received heart rate to the end of the transition of the heart rate. The fatigue level estimation unit 120 may estimate the fatigue level as described above based on the transition of the heart rate held. In this case, receiving a plurality of heart rates is related to receiving transition of heart rate. Then, in this case, instead of step S101 in FIG. 6, the reception unit 110 receives the heart rate, and the fatigue level estimation unit 120 updates the transition of the heart rate held using the received heart rate. FIG. 7 described below illustrates an example of the operation of the estimation device 100 in this case.

FIG. 7 is a flowchart illustrating an example of the operation of the estimation device 100 according to a modified example of the second example embodiment of the present disclosure.

The operation illustrated in FIG. 7 is different from the operation illustrated in FIG. 6 in that the operation of step S201 is performed instead of step S101. In step S201, the estimation device 100 performs the receiving process. The estimation device 100 performs the same operation as the operation after step S102 illustrated in FIG. 6 in the operation after step S102. The estimation device 100 repeats the operation of FIG. 7 every time the heart rate is received from the heart rate measurement device 200.

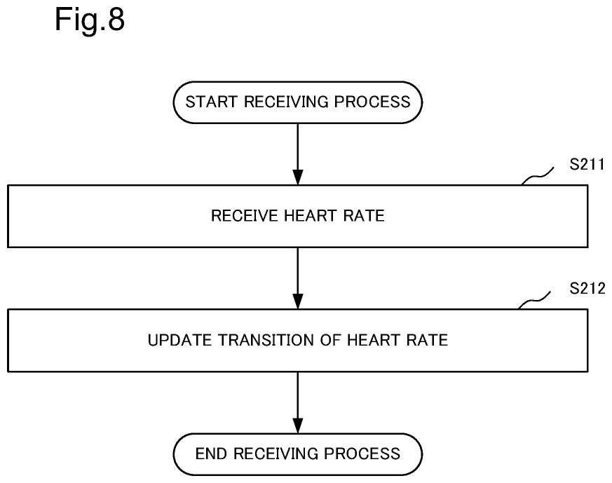

FIG. 8 is a flowchart illustrating an operation of the receiving process of the estimation device 100 according to the second example embodiment of the present disclosure. In the example illustrated in FIG. 8, the reception unit 110 receives the heart rate from the heart rate measurement device 200 (step S211). Specifically, the reception unit 110 receives information indicating the heart rate from the heart rate measurement device 200. Next, the fatigue level estimation unit 120 updates the transition of the heart rate using the received heart rate (step S212). Specifically, the fatigue level estimation unit 120 updates the transition of the heart rate by adding the received heart rate to the end of the transition of the heart rate.

### <Effects>

The present example embodiment has the same effect as that of the first example embodiment. The reason is the same as the reason why the effect of the first example embodiment occurs.

The present example embodiment has an effect of preventing overwork and the like of the target person. This is because the notification unit 160 makes a notification when the fatigue level of the target person indicates that the fatigue is greater than a predetermined level.

### <Fatigue Level and Exercise Intensity>

Hereinafter, the fatigue level in the description of the present disclosure will be described in detail.

As the fatigue level, a Borg scale fatigue level is known. The Borg scale is an index for subjectively evaluating own sensation during exercise of a subject, which is also recognized in clinical medicine in consideration of physical strength, environment, and general fatigue factors of an individual. The Borg scale fatigue level, which is an example of the Borg scale, is an index representing the degree of fatigue in 15 stages, one stage being 10 beats of the heartbeat, assuming that the resting heart rate is 60 and the maximum estimated heart rate is 220. The maximum estimated heart rate is a heart rate estimated as a maximum value of the heart rate. The Borg scale fatigue level is known as an exercise evaluation index suitable for actual exercise measurement. There is also a modified Polg index for explaining a non-linear index such as the change amount of the blood lactic acid value and the oxygen saturation. The stages of the Borg scale fatigue level are determined based on the ratio of the heart rate with respect to the maximum heart rate. In the Borg scale fatigue level, it is assumed that the heart rate in a slightly tight state is a heart rate of 60% of the maximum estimated heart rate, and the heart rate in a tight state is a heart rate of 85% of the maximum estimated heart rate. The relationship between the heart rate and the ratio of the heart rate with respect to the maximum estimated heart rate used in the Borg scale fatigue level is expressed by the following equation.

$$\hat{X} = ( 220 - a ) \times {Y}_{Borg} [ \% ] / 100 \qquad [ Math . 1 ]$$

In Math. 1, a represents the age of the target person, Y_{Borg} [%] represents the ratio of the heart rate with respect to the maximum estimated heart rate expressed in units of percent, and X\hat{ } (i.e., a variable having a hat symbol above X) represents the heart rate. With this equation, the heart rate X\hat{ } of the individual when Y_{Borg} [%] is designated can be estimated.

In the following description, Y_{Borg} [%] is referred to as a Borg index. Since the heart rate X\hat{ } of the individual when Y_{Borg} [%] is designated can be estimated by the equation shown in Math. 1, X\hat{ } is also referred to as an estimated heart rate.

The equation of Math. 1 can be transformed to the following equation.

$${Y}_{Borg} [ \% ] = \frac{100}{( 220 - a )} \times \hat{X} = {k}_{age} \times \hat{X} \qquad [ Math . 2 ]$$

In the equation of Math. 2, k_{age} corresponds to the slope of a straight line determined by age. The slope k_{age} becomes smaller as the age becomes lower.

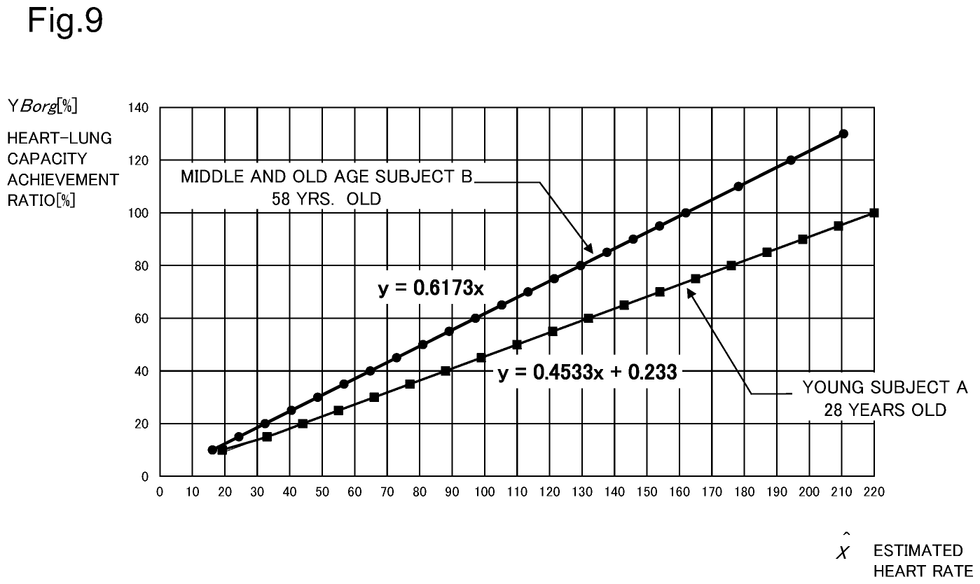

FIG. 9 is a diagram illustrating an example of Borg index with respect to the estimated heart rate. FIG. 9 illustrates the relationship between the estimated heart rates of two people with different ages and the Borg index.

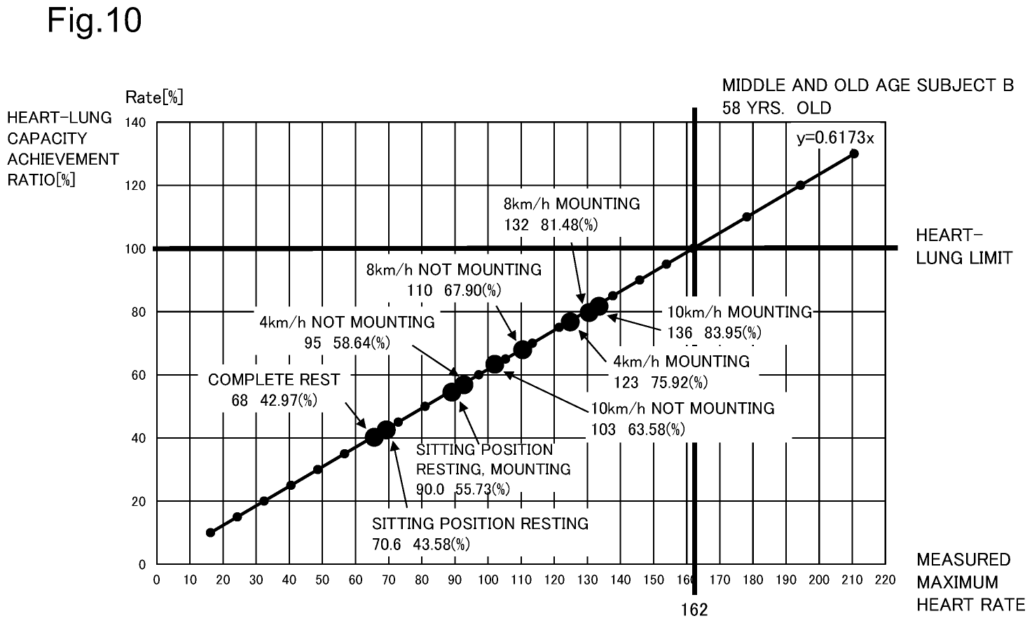

FIG. 10 is a diagram illustrating an example of a relationship between the measured maximum heart rate and the heart-lung capacity achievement ratio. The heart-lung capacity achievement ratio represents a heart-lung capacity achievement ratio which is a ratio of a heart rate with respect to a heart-lung limit when a heart rate obtained by subtracting age from 220 is regarded as a limit of the heart-lung capacity (hereinafter, also referred to as heart-lung limit). In FIG. 10, heart rates measured under various conditions are plotted. “Mounting” in FIG. 10 indicates that the measurement is performed in a state where the walking assist robot is mounted. “Not mounting” indicates that the measurement is performed in a state where the walking assist robot is not mounted.

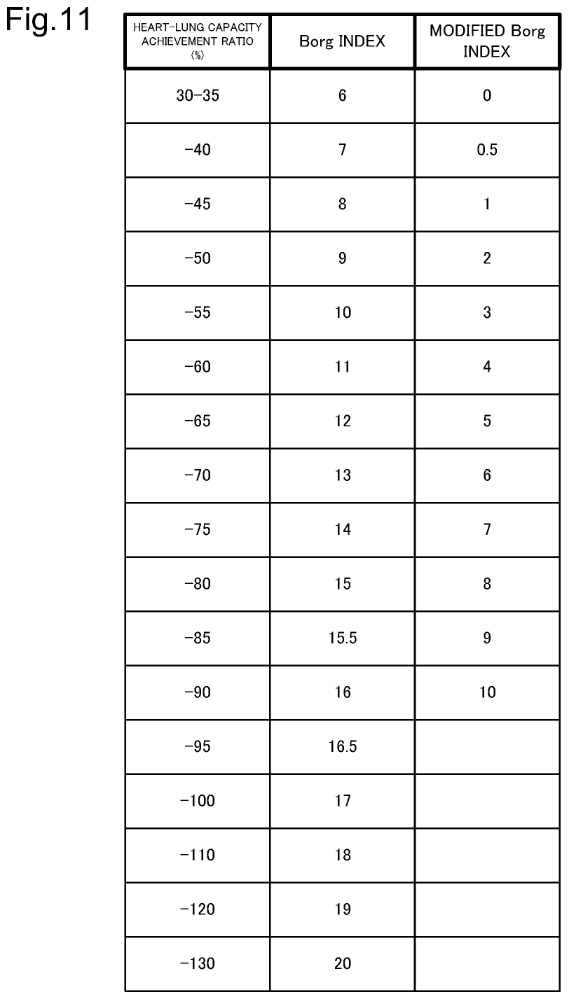

FIG. 11 is a diagram illustrating another example of the relationship between the measured maximum heart rate and the heart-lung capacity achievement ratio.

Since the subjective index indicated by the Borg index with respect to the measured maximum heart rate by the target person at the time of measuring the heart rate illustrated in FIGS. 10 and 11 substantially coincides with the subjective state of awareness, the Borg index is estimated as realizing an appropriate subjective index of the subjective symptom to some extent.

In the equations of Math. 1 and Math. 2, in a case where expansion is performed using X\hat{ } and Y_{Borg} [%] as the heart rate measured at time t and the Borg index, Math. 1 is expressed as the following equation.

$$\hat{X} ( t ) = ( 220 - a ) \times {Y}_{Borg} ( t ) [ \% ] / 100 \qquad [ Math . 3 ]$$

Furthermore, Y_{Borg} (t) [%] is a ratio of the heart-lung capacity at the time of heart rate measured at time t with respect to the heart-lung capacity at the time when the maximum estimated heart rate is observed, with the resting heart rate as a baseline in the range of the observed maximum heart rate and the resting heart rate according to the definition of the Borg scale. Y_{Borg} (t) [%] is also referred to as a heart-lung capacity achievement ratio or a maximum heart-lung capacity achievement ratio.

Therefore, Y_{Borg} (t) [%] is equivalent to the heart-lung capacity contrast of the heart rate at time t in the maximum observed heart rate range based on the resting heart rate before exercise. Hereinafter, the observed maximum heart rate is denoted as Xₘₐₓ, and the observed resting heart rate is denoted as Xₘᵢₙ. Y_{Borg}(t) [%] is expressed by the following equation.

$$\begin{matrix}{Y}_{Borg} ( t ) [ \% ] = \frac{\begin{matrix}\begin{matrix}( HEART RATE AT TIME OF \\ EXERCISE AT TIME t -\end{matrix} \\ RESTING HEART RATE )\end{matrix}}{\begin{matrix}( MAXIMUM OBSERVED HEART RATE + \\ RESTING HEART RATE )\end{matrix}} \times \\ 100 [ \% ] \\ = \frac{x ( t ) - {x}_{min}}{{x}_{max} + {x}_{min}} \times 100 [ \% ]\end{matrix} \qquad [ Math . 4 ]$$

In the equation of Math. 4, x(t) represents the heart rate measured at time t.

Meanwhile, the aerobic exercise intensity VO₂ₘₐₓ, which is two exercise intensities, at time t, is expressed as the following equation from the definition thereof.

$$\begin{matrix}{VO}_{2 max} ( t ) = \frac{\begin{matrix}\begin{matrix}( HEART RATE AT TIME OF \\ EXERCISE AT TIME t -\end{matrix} \\ RESTING HEART RATE )\end{matrix}}{\begin{matrix}( MAXIMUM OBSERVED HEART RATE - \\ RESTING HEART RATE )\end{matrix}} \times \\ 100 [ \% ] \\ = \frac{x ( t ) - {x}_{min}}{{x}_{max} - {x}_{min}} \times 100 [ \% ]\end{matrix} \qquad [ Math . 5 ]$$

The following equation is established from Math. 2 and Math. 4.

$$\begin{matrix}\hat{X} ( t ) = ( 220 - a ) \times \frac{{Y}_{Borg} ( t )}{100} \\ = \frac{( 220 - a )}{100} \times \frac{x ( t ) - {x}_{min}}{{x}_{max} + {x}_{min}} \times 100 [ \% ]\end{matrix} \qquad [ Math . 6 ]$$

The numerator and the denominator on the right side of Math. 6 is divided by xₘₐₓ−xₘᵢₙ, respectively, to obtain the following equation.

$$\begin{matrix}\hat{X} ( t ) = ( 220 - a ) \times \frac{( \frac{x ( t ) - {x}_{min}}{{x}_{max} - {x}_{min}} )}{( \frac{{x}_{max} + {x}_{min}}{{x}_{max} - {x}_{min}} )} \\ = ( 220 - a ) \times \frac{( \frac{x ( t ) - {x}_{min}}{{x}_{max} - {x}_{min}} )}{( \frac{{x}_{max} - {x}_{min} + 2 \times {x}_{min}}{{x}_{max} - {x}_{min}} )}\end{matrix} \qquad [ Math . 7 ]$$

When the equation of Math. 7 is expressed by VO₂ₘₐₓ shown in Math. 5, the following equation is obtained.

$$\hat{X} ( t ) = ( 220 - a ) \times \frac{{VO}_{2 max} ( t )}{1 + \frac{2 \times {x}_{min}}{{x}_{max} - {x}_{min}}} \qquad [ Math . 8 ]$$

The following equation is a parameter used to deform the equation of Math. 8.

$$α = 220 - a \qquad [ Math . 9 ]$$

$$β = \frac{2 \times {x}_{min}}{{x}_{max} - {x}_{min}}$$

The following equation is an equation obtained by rewriting the equation of Math. 8 using the parameters shown in Math. 9.

$$\hat{X} ( t ) = \frac{α \times {VO}_{2 max} ( t )}{1 + β} \qquad [ Math . 10 ]$$

An equation obtained by deforming the equation of Math. 10 so that VO₂ₘₐₓ(t) is on the left side is the following expression.

$${VO}_{2 max} ( t ) = ( \frac{1 + β}{α} ) \times \hat{X} ( t ) = k \times \hat{X} ( t ) \qquad [ Math . 11 ]$$

As shown in Math. 11, the aerobic exercise intensity VO₂ₘₐₓ [%] can be expressed with a simple linear function determined by an age parameter by a coefficient α and the heart-lung capacity (maximum observed heart rate xₘₐₓ and the resting heart rate xₘᵢₙ) of an individual with the Borg estimated heart rate x(t) as a variable. By performing calculation according to the equations of Math. 4 and Math. 11, it is possible to quantify the exercise intensity considering not only the age condition like the conventional Borg index but also the heart-lung capacity of the individual, and to perform subjective evaluation based on the Borg index in real time.

The fatigue level estimation unit 120 estimates the fatigue level according to Math. 4. Specifically, the fatigue level estimation unit 120 detects the maximum value (i.e., xₘₐₓ) of the heart rate in the transition of the heart rate as the measured maximum heart rate, and detects the minimum value (i.e., xₘᵢₙ) of the heart rate in the transition of the heart rate as the resting heart rate. Using the heart rate x(t) at the fatigue level estimation target time point (i.e., time t), the fatigue level estimation unit 120 calculates the heart-lung capacity achievement ratio Y_{Borg}(t) according to the equation of Math. 4. The fatigue level estimation unit 120 sets the fatigue level corresponding to the calculated heart-lung capacity achievement ratio Y_{Borg} (t) as the fatigue level at the fatigue level estimation target time point.

FIG. 11 is a diagram illustrating a relationship between the heart-lung capacity achievement ratio and an example of an index. FIG. 11 illustrates the relationship between the heart-lung capacity achievement ratio and the Borg index and the modified Borg index. For example, the fatigue level estimation unit 120 sets, as the fatigue level, an index (e.g., Borg index or modified Borg index) corresponding to a range including the calculated local ability achievement ratio.

For example, the exercise intensity estimation unit 140 calculates VO₂ₘₐₓ (t) according to the equation of FIG. 11, and sets the calculated aerobic exercise intensity VO₂ₘₐₓ (t) as the exercise intensity. The exercise intensity estimation unit 140 may first calculate the parameter k in the equation of Math. 10 and hold the calculated parameter k. Then, the exercise intensity estimation unit 140 may calculate the aerobic exercise intensity VO₂ₘₐₓ (t) using the held parameter k and the heart rate (X\hat{ } (t)) at the intensity estimation target time point (i.e., time t).

### <Stabilization Time>

In the above description of the stabilization time estimation unit 150, the transition of the heart rate is measured so as to include the transition of the heart rate measured until the target person becomes the resting state in the state of the maximum heart rate and the heart rate of the target person becomes the resting heart rate. However, even in a case where the transition of the heart rate is not measured as described above, for example, the stabilization time estimation unit 150 can estimate the stabilization time in the following manner.

The stabilization time estimation unit 150 detects a maximum value and a minimum value of the heart rate in the transition of the heart rate. The stabilization time estimation unit 150 calculates the slope of the change in the heart rate between the detected maximum value and the minimum value detected next in the time direction from the time when the maximum value is observed. The stabilization time estimation unit 150 may calculate the slope of the change in the heart rate between the detected maximum value and the minimum value detected next after elapse of a predetermined time or more from the time when the maximum value is observed. The stabilization time estimation unit 150 may smooth the transition of the heart rate and detect the maximum value and the minimum value in the transition of the heart rate after the smoothing. When a plurality of slopes are calculated, the stabilization time estimation unit 150 calculates a statistical value (e.g., an average value, an intermediate value, a median value, etc.) of the calculated slopes.

The stabilization time estimation unit 150 calculates a time until the heart rate reaches the heart rate in the sitting position stable state in a case where the heart rate decreases at the decrease rate represented by the calculated slope from the heart rate (x (t)) measured at the stabilization time estimation target time point (time t). The stabilization time estimation unit 150 may set the minimum value of the heart rate in the transition of the heart rate as the heart rate in the sitting position stable state.

The stabilization time estimation unit 150 may apply the transition of the heart rate between the maximum value and the minimum value to an equation (e.g., a polynomial of time) other than the straight line. Specifically, the stabilization time estimation unit 150 calculates a parameter of a polynomial representing the transition of the heart rate between the maximum value and the minimum value. The stabilization time estimation unit 150 calculates a time until the heart rate reaches the heart rate in the sitting stable state in a case where the heart rate decreases according to a polynomial based on the calculated parameter from the heart rate (x (t)) measured at the stabilization time estimation target time point (time t).

The stabilization time estimation unit 150 can also calculate the stabilization time based on the transition of the exercise intensity calculated from the transition of the heart rate. The stabilization time estimation unit 150 detects the maximum value and the minimum value in the transition of the exercise intensity, calculates the slope as described above, and calculates the statistical value of the slope. The stabilization time estimation unit 150 calculates the time until the exercise intensity becomes zero as the stabilization time in a case where the exercise intensity decreases from the value of the exercise intensity calculated from the heart rate (x (t)) measured at the stabilization time estimation target time point (time t) according to the statistical value of the calculated slope.

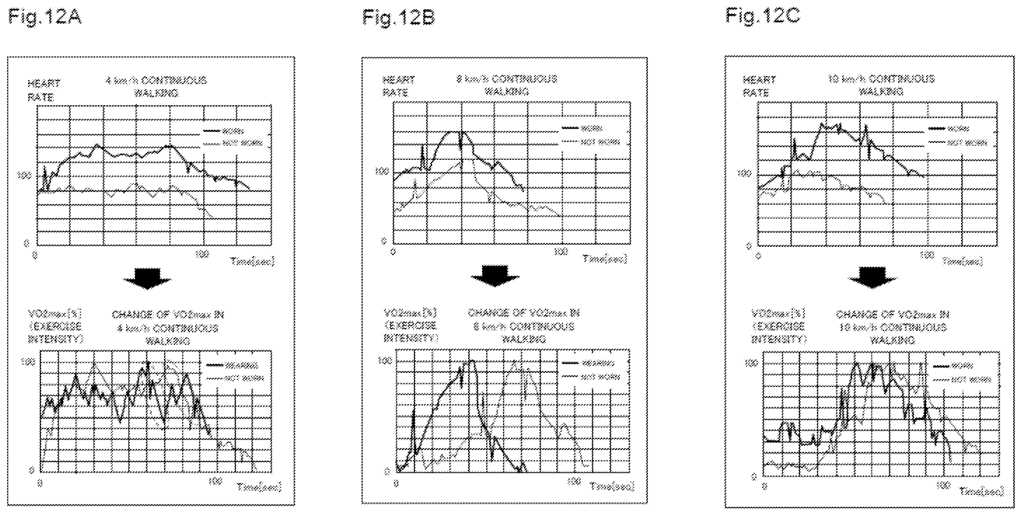

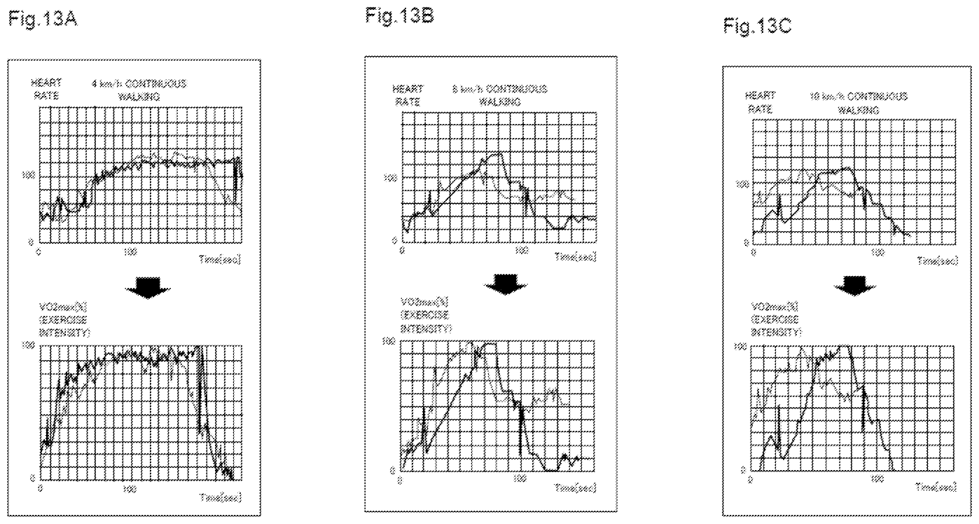

FIGS. 12 and 13 are diagrams illustrating the transition of the heart rate and the transition of the exercise intensity. FIGS. 12 and 13 illustrate the transition of the heart rate and the transition of the exercise intensity in a case where a walking assist suit (also referred to as a robot suit) is worn and in a case where the walking assist suit is not worn.

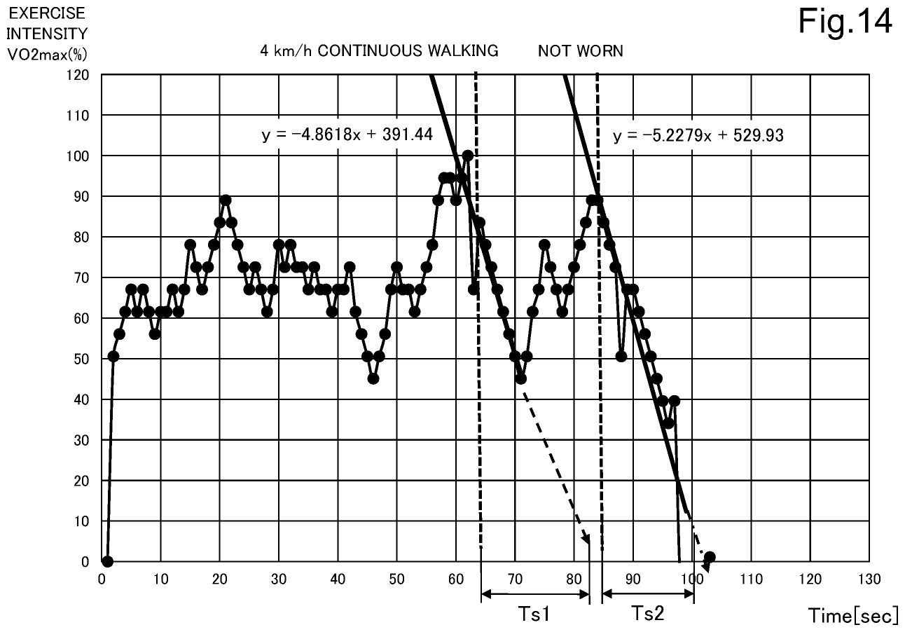

FIG. 14 is a diagram illustrating an example of a transition of the heart rate and an example of a straight line of the calculated slope. In the example illustrated in FIG. 14, the slope of the straight line is calculated at two points. The stabilization times calculated from the two slopes are Tₛ₁ and Tₛ₂.

### <Effects>

Effects of the second example embodiment of the present disclosure will be further described.

As described above, it can be seen that the feature amount of the relationship between the time series at the time of exercise and the heart rate remarkably appears in the exercise intensity [%]. Furthermore, the subjective symptom of the Borg scale and the VO2max numerical information can be displayed in real time only with the observation data y(t) of the heart rate. This has an effect that not only the subject himself/herself who is exercising but also the observer can easily grasp the physical power margin and the subjective symptom of the subject, and whether the subject has reached the excessive load on the heart-lung and the exercise can be easily determined. There are effects of heart-lung capacity and residual heart-lung capacity at the exercise time t.

Furthermore, the stabilization time required from the maximum heart rate observation to the sitting position stability is not determined by the amount of exercise (length of time of exercise) or the load (robot load), but the relationship between the stabilization time until returning to the heart rate at the resting sitting position from the maximum heart rate observation and the exercise intensity [%] is substantially linear under the same walking speed condition, and when approximated with a polynomial having time t as a variable, the stabilization time (Ts) having the maximum observation time as the starting point can be easily estimated. The reciprocal of the stabilization time can be expressed in a numerical form as the speed of the recovery degree of the subject (strength of the physical strength recovery degree).

When the relationship between the exercise intensity expressed by VO2max and the estimated heart rate based on the Borg index is obtained, the slope information of the straight line is equivalent to the numerical information on the degree of the load, and the high and low of the load directly connected to fatigue can be quantified as a numerical value by comparing the slope information of the straight line. By incorporating these relational expressions into the system and cooperatively operating them with the wearable heart rate meter, in the case of the same subject, the exercise intensity at that time is obtained only by the heart rate measured in real time, so that an effect is obtained in that the current usage degree with respect to the maximum heart-lung capacity of the individual can be quantitatively grasped.

In addition, although only the age parameter considered the personal information in the Borg index so far, in the present example embodiment, the extended Borg index can calculate the heart-lung capacity ratio at time t with respect to the maximum heart-lung capacity of the individual from only the maximum heart rate and the resting heart rate data of the individual and the heart rate at time t, and thus an effect is obtained in that the heart-lung capacity of the individual closer to the current state of the heart-lung capacity of the individual can be grasped based on the heart-lung capacity ratio determined in advance by the Borg index. Furthermore, an effect is obtained in that the subjective fatigue awareness at time t can be displayed based on the heart-lung ratio determined by the Borg index, and a third person other than the subject can also easily grasp the fatigue level during exercise of the subject.

### Other Example Embodiments

The estimation device 10 and the estimation device 100 described above can be achieved by a computer including a memory in which a program read from a storage medium is loaded and a processor that executes the program. The estimation device 10 and the estimation device 100 can also be achieved by dedicated hardware. The estimation device 10 and the estimation device 100 can also be achieved by a combination of the above-described computer and dedicated hardware.

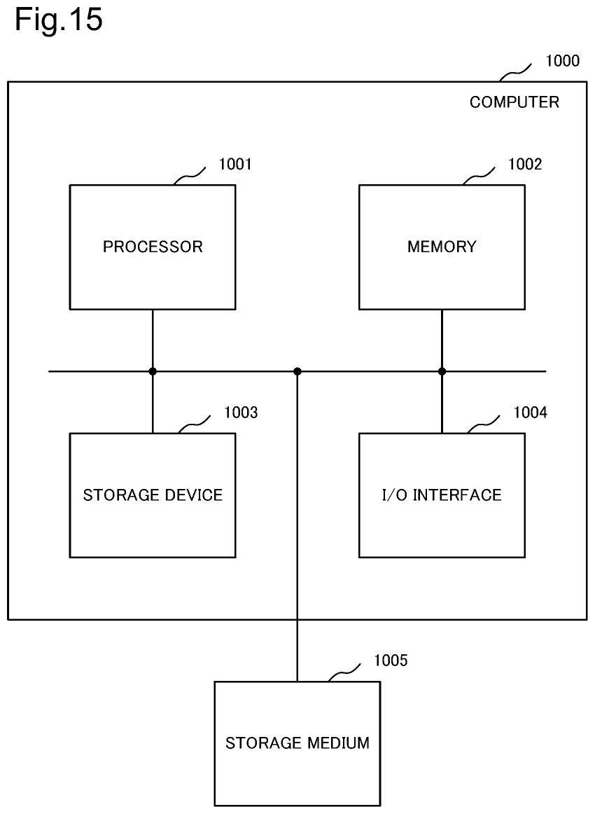

FIG. 15 is a diagram illustrating an example of a hardware configuration of a computer 1000 that can realize each of the estimation devices according to the example embodiments of the present disclosure. In the example illustrated in FIG. 15, the computer 1000 includes a processor 1001, a memory 1002, a storage device 1003, and an input/output (I/O) interface 1004. In addition, the computer 1000 can access the storage medium 1005. The memory 1002 and the storage device 1003 are, for example, storage devices such as a random access memory (RAM) and a hard disk. The storage medium 1005 is, for example, a storage device such as a RAM or a hard disk, a read only memory (ROM), or a portable storage medium. The storage device 1003 may be the storage medium 1005. The processor 1001 can read and write data and programs from and to the memory 1002 and the storage device 1003. The processor 1001 can access, for example, the heart rate measurement device 200, the output device 300, and the notification device 400 via the I/O interface 1004. The processor 1001 may access the storage medium 1005. The storage medium 1005 stores a program for operating the computer 1000 as the estimation device according to the example embodiment of the present disclosure.

The processor 1001 loads a program, which is stored in the storage medium 1005 and causes the computer 1000 to operate as the estimation device according to the example embodiment of the present disclosure, into the memory 1002. Then, when the processor 1001 executes the program loaded in the memory 1002, the computer 1000 operates as the estimation device according to the example embodiment of the present disclosure.

The reception unit 110, the fatigue level estimation unit 120, the output unit 130, the exercise intensity estimation unit 140, the stabilization time estimation unit 150, and the notification unit 160 can be achieved by, for example, the processor 1001 that executes a program loaded in the memory 1002. Some or all of the reception unit 110, the fatigue level estimation unit 120, the output unit 130, the exercise intensity estimation unit 140, the stabilization time estimation unit 150, and the notification unit 160 can be achieved by a dedicated circuit that realizes the function of each unit.

Furthermore, some or all of the above example embodiments may be described as the following supplementary notes, but are not limited to the following.

### (Supplementary Note 1)

An estimation device including:
reception means for receiving a transition of a heart rate of a target person in a state including a resting state and an active state, the transition of the heart rate being measured by a heart rate measurement device;
fatigue level estimation means for estimating a fatigue level of the target person based on an estimation model and the transition of the heart rate, the estimation model estimating a fatigue level based on the heart rate; and
output means for outputting the fatigue level.

### (Supplementary Note 2)

The estimation device according to supplementary note 1, wherein
the estimation model estimates the fatigue level based on a measured maximum heart rate and a resting heart rate, the measured maximum heart rate being a maximum heart rate in the transition of the heart rate, the resting heart rate being a heart rate in the resting state.

### (Supplementary Note 3)

The estimation device according to supplementary note 2, wherein
the estimation model estimates, based further on a heart rate at a fatigue level estimation target time point, the fatigue level at the fatigue level estimation target time point.

### (Supplementary Note 4)

The estimation device according to any one of supplementary notes 1 to 3, further including
exercise intensity estimation means for estimating an exercise intensity at an intensity estimation target time point based on the transition of the heart rate, wherein
the output means further outputs the exercise intensity.

### (Supplementary Note 5)

The estimation device according to supplementary note 4, wherein
the fatigue level estimation means estimates, based on a latest heart rate, the fatigue level at a time point when the latest heart rate is measured,
the exercise intensity estimation means estimates, based on the latest heart rate, the exercise intensity at the time point when the latest heart rate is measured, and
the output means outputs the fatigue level and the exercise intensity at the time point when the latest heart rate is measured.

### (Supplementary Note 6)

The estimation device according to any one of supplementary notes 1 to 5, further including
stabilization time estimation means for estimating a stabilization time in a case where the state of the target person transitions to the resting state at a stabilization time estimation target time point based on the transition of the measured heart rate, the stabilization time being a time from the stabilization time estimation target time point until the heart rate of the target person reaches the resting state.

### (Supplementary Note 7)

The estimation device according to any one of supplementary notes 1 to 6, further including
notification means for performing a notification when the fatigue level indicates that fatigue is greater than a predetermined level.

### (Supplementary Note 8)

An estimation system including the estimation device described in any one of supplementary notes 1 to 7, comprising:
the heart rate measurement device.

### (Supplementary Note 9)

An estimation method including:
receiving a transition of a heart rate of a target person in a state including a resting state and an active state, the transition of the heart rate being measured by a heart rate measurement device;
estimating a fatigue level of the target person based on an estimation model and the transition of the heart rate, the estimation model estimating a fatigue level based on the heart rate; and
outputting the fatigue level.

### (Supplementary Note 10)

The estimation method according to supplementary note 9, wherein
the estimation model estimates the fatigue level based on a measured maximum heart rate and a resting heart rate, the measured maximum heart rate being a maximum heart rate in the transition of the heart rate, the resting heart rate being a heart rate in the resting state.

### (Supplementary Note 11)

The estimation method according to supplementary note 10, wherein
the estimation model estimates, based further on a heart rate at a fatigue level estimation target time point, the fatigue level at the fatigue level estimation target time point.

### (Supplementary Note 12)

The estimation method according to any one of supplementary notes 9 to 11, further including:
estimating an exercise intensity at an intensity estimation target time point based on the transition of the heart rate; and
further outputting the exercise intensity.

### (Supplementary Note 13)

The estimation method according to supplementary note 12, further including:
estimating, based on a latest heart rate, the fatigue level at a time point when the latest heart rate is measured,
estimating, based on the latest heart rate, the exercise intensity at the time point when the latest heart rate is measured, and
outputting, the fatigue level and the exercise intensity at the time point when the latest heart rate is measured.

### (Supplementary Note 14)

The estimation method according to any one of supplementary notes 9 to 13, further including
estimating a stabilization time in a case where the state of the target person transitions to the resting state at a stabilization time estimation target time point based on the transition of the measured heart rate, the stabilization time being a time from the stabilization time estimation target time point until the heart rate of the target person reaches the resting state.

### (Supplementary Note 15)

The estimation method according to any one of supplementary notes 9 to 14, further including
performing a notification when the fatigue level indicates that fatigue is greater than a predetermined level.

### (Supplementary Note 16)

A storage medium storing a program that causes a computer to execute:
reception processing of receiving a transition of a heart rate of a target person in a state including a resting state and an active state, the transition of the heart rate being measured by a heart rate measurement device;
fatigue level estimation processing of estimating a fatigue level of the target person based on an estimation model and the transition of the heart rate, the estimation model estimating a fatigue level based on a heart rate; and
output processing of outputting the fatigue level.

### (Supplementary Note 17)

The storage medium according to supplementary note 16, wherein
the estimation model estimates the fatigue level based on a measured maximum heart rate and a resting heart rate, the measured maximum heart rate being a maximum heart rate in the transition of the heart rate, the resting heart rate being a heart rate in the resting state.

### (Supplementary Note 18)

The storage medium according to supplementary note 17, wherein
the estimation model estimates, based further on a heart rate at a fatigue level estimation target time point, the fatigue level at the fatigue level estimation target time point.

### (Supplementary Note 19)

The storage medium according to any one of supplementary notes 16 to 18, wherein the program causes the computer to execute
exercise intensity estimation processing of estimating an exercise intensity at an intensity estimation target time point based on the transition of the heart rate, and the output process further outputs the exercise intensity.

### (Supplementary Note 20)

The storage medium according to supplementary note 19, wherein
the fatigue level estimation processing estimates, based on a latest heart rate, the fatigue level at a time point when the latest heart rate is measured,
the exercise intensity estimation processing estimates, based on the latest heart rate, the exercise intensity at the time point when the latest heart rate is measured, and
the output processing outputs the fatigue level and the exercise intensity at the time point when the latest heart rate is measured.

### (Supplementary Note 21)

The storage medium according to any one of supplementary notes 16 to 20, where the program causes the computer to execute
stabilization time estimation processing of estimating a stabilization time in a case where the state of the target person transitions to the resting state at a stabilization time estimation target time point based on the transition of the measured heart rate, the stabilization time being a time from the stabilization time estimation target time point until the heart rate of the target person reaches the resting state.

### (Supplementary Note 22)

The storage medium according to any one of supplementary notes 16 to 21, wherein the program further causes the computer to execute
notification processing of performing a notification in a case where the fatigue level indicates that fatigue is greater than a predetermined level.

While the invention has been particularly shown and described with reference to example embodiments thereof, the invention is not limited to these example embodiments. It will be understood by those of ordinary skill in the art that various changes in form and details may be made therein without departing from the spirit and scope of the present invention as defined by the claims.

### REFERENCE SIGNS LIST

1 Estimation system
10 Estimation device
100 Estimation device
110 Reception unit
120 Fatigue level estimation unit
130 Output unit
140 Exercise intensity estimation unit
150 Stabilization time estimation unit
160 Notification unit
200 Heart rate measurement device
300 Output device
400 Notification device
500 Wearable device
1000 Computer
1001 Processor
1002 Memory
1003 Storage device
1004 I/O interface
1005 Storage medium

What is claimed is:

1. An estimation device comprising:
at least one memory storing a set of instructions; and
at least one processor configured to execute the set of instructions to:
receive a transition of a heart rate of a target person in a state including a resting state and an active state, the transition of the heart rate being measured by a heart rate measurement device;
estimate a fatigue level of the target person based on an estimation model and the transition of the heart rate, the estimation model estimating the fatigue level based on the heart rate; and
output the fatigue level.

2. The estimation device according to claim 1, wherein
the estimation model estimates the fatigue level based on a measured maximum heart rate and a resting heart rate, the measured maximum heart rate being a maximum heart rate in the transition of the heart rate, the resting heart rate being a heart rate in the resting state.

3. The estimation device according to claim 2, wherein
the estimation model estimates, based further on a heart rate at a fatigue level estimation target time point, the fatigue level at the fatigue level estimation target time point.

4. The estimation device according to claim 1, wherein
the at least one processor is further configured to execute the instructions to:
estimate an exercise intensity at an intensity estimation target time point based on the transition of the heart rate; and
output the exercise intensity.

5. The estimation device according to claim 4, wherein
the at least one processor is further configured to execute the instructions to:
estimate, based on a latest heart rate, the fatigue level at a time point when the latest heart rate is measured;
estimate, based on the latest heart rate, the exercise intensity at the time point when the latest heart rate is measured; and
output the fatigue level and the exercise intensity at the time point when the latest heart rate is measured.

6. The estimation device according to claim 1, wherein
the at least one processor is further configured to execute the instructions to
estimate a stabilization time in a case where the state of the target person transitions to the resting state at a stabilization time estimation target time point based on the transition of the measured heart rate, the stabilization time being a time from the stabilization time estimation target time point until the heart rate of the target person reaches the resting state.

7. The estimation device according to claim 1, wherein
the at least one processor is further configured to execute the instructions to
perform a notification when the fatigue level indicates that fatigue is greater than a predetermined level.

8. An estimation system including the estimation device according to claim 1, comprising:
the heart rate measurement device.

9. An estimation method comprising:
receiving a transition of a heart rate of a target person in a state including a resting state and an active state, the transition of the heart rate being measured by a heart rate measurement device;
estimating a fatigue level of the target person based on an estimation model and the transition of the heart rate, the estimation model estimating a fatigue level based on the heart rate; and
outputting the fatigue level.

10. The estimation method according to claim 9, wherein
the estimation model estimates the fatigue level based on a measured maximum heart rate and a resting heart rate, the measured maximum heart rate being a maximum heart rate in the transition of the heart rate, the resting heart rate being a heart rate in the resting state.

11. The estimation method according to claim 10, wherein
the estimation model estimates, based further on a heart rate at a fatigue level estimation target time point, the fatigue level at the fatigue level estimation target time point.

12. The estimation method according to claim 9, further comprising:
estimating an exercise intensity at an intensity estimation target time point based on the transition of the heart rate; and
further outputting the exercise intensity.

13. The estimation method according to claim 12, further comprising:
estimating, based on a latest heart rate, the fatigue level at a time point when the latest heart rate is measured,
estimating, based on the latest heart rate, the exercise intensity at the time point when the latest heart rate is measured, and
outputting, the fatigue level and the exercise intensity at the time point when the latest heart rate is measured.

14. The estimation method according to claim 9, further comprising:
estimating a stabilization time in a case where the state of the target person transitions to the resting state at a stabilization time estimation target time point based on the transition of the measured heart rate, the stabilization time being a time from the stabilization time estimation target time point until the heart rate of the target person reaches the resting state.

15. The estimation method according to claim 9, further comprising
performing a notification when the fatigue level indicates that fatigue is greater than a predetermined level.

16. A non-transitory computer readable storage medium storing a program that causes a computer to execute:
reception processing of receiving a transition of a heart rate of a target person in a state including a resting state and an active state, the transition of the heart rate being measured by a heart rate measurement device;
fatigue level estimation processing of estimating a fatigue level of the target person based on an estimation model and the transition of the heart rate, the estimation model estimating the fatigue level based on the heart rate; and
output processing of outputting the fatigue level.

17. The non-transitory computer readable storage medium according to claim 16, wherein
the estimation model estimates the fatigue level based on a measured maximum heart rate and a resting heart rate, the measured maximum heart rate being a maximum heart rate in the transition of the heart rate, the resting heart rate being a heart rate in the resting state.

18. The non-transitory computer readable storage medium according to claim 17, wherein
the estimation model estimates, based further on a heart rate at a fatigue level estimation target time point, the fatigue level at the fatigue level estimation target time point.

19. The non-transitory computer readable storage medium according to claim 16, wherein
the program causes the computer to execute
exercise intensity estimation processing of estimating an exercise intensity at an intensity estimation target time point based on the transition of the heart rate, and
the output processing further outputs the exercise intensity.

20. The non-transitory computer readable storage medium according to claim 19, wherein
the fatigue level estimation processing estimates, based on a latest heart rate, the fatigue level at a time point when the latest heart rate is measured,
the exercise intensity estimation processing estimates, based on the latest heart rate, the exercise intensity at the time point when the latest heart rate is measured, and
the output processing outputs the fatigue level and the exercise intensity at the time point when the latest heart rate is measured.

21-22. (canceled)
